(19) United States
(12) Patent Application Publication
(10) Pub. No.: US 2010/0227856 A1
(43) Pub. Date: Sep. 9, 2010

(54) NOVEL COMPOUNDS

(75) Inventor: Robert W. Marquis, JR., Collegeville, PA (US)

Correspondence Address: GlaxoSmithKline;GLOBAL PATENTS -US, UW2220, P. O. BOX 1539, KING OF PRUSSIA, PA, 19406-0939, US

(73) Assignee: SMITHKLINE BEECHAM CORPORATION, Philadelphia, PA (US)

(21) Appl. No.: 12/279,562

(22) PCT Filed: Feb. 16, 2007
(86) PCT No.: PCT/US2007/062297

§ 371 Date: Aug. 15, 2008

Related U.S. Application Data
(60) Provisional application No. 60/774,386, filed on Feb. 17, 2006.

(51) Int. Cl. A61K 31/541; A61K 31/4535; C07D 417/12; C07D 409/12; A61P 25/28; A61P 19/00; A61P 19/02; A61P 19/04; A61P 29/00

(52) U.S. Cl. 514/228.2; 514/324; 544/58.2; 544/58.7; 546/202

(57) ABSTRACT

This invention relates to novel compounds useful in the treatment of diseases associated with TRPV4 channel receptor. More specifically, this invention relates to certain substituted piperidines, according to Formula I

Specifically, the invention is directed to compounds according to Formula I

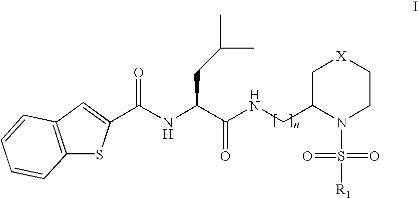

wherein
R¹ is optionally substituted aryl;
X is CH₂, S, or SO₂; and
n=1 or 2.

### FIELD OF THE INVENTION

This invention relates to novel compounds useful in the treatment of diseases associated with TRPV4 channel receptor. More specifically, this invention relates to certain substituted piperidines, which are agonists of TRPV4 channel receptors.

### BACKGROUND OF THE INVENTION

Cartilage is an avascular tissue populated by specialized cells termed chondrocytes, which respond to diverse mechanical and biochemical stimuli. Cartilage is present in the linings of joints, interstitial connective tissues, and basement membranes, and is composed of an extracellular matrix comprised of several matrix components including type II collagen, proteoglycans, fibronectin and laminin.

In normal cartilage, extracellular matrix synthesis is offset by extracellular matrix degradation, resulting in normal matrix turnover. Depending on the signal(s) received, the ensuing response may be either anabolic (leading to matrix production and/or repair) or catabolic (leading to matrix degradation, cellular apoptosis, loss of function, and pain).

TRPV4 channel receptor is one of six known members of the vanilloid family of transient receptor potential channels and shares 51% identity at the nucleotide level with TRPV1, the capsaicin receptor. Examples of polypeptides and polynucleotides encoding forms of human vanilloid receptors, including TRPV4 channel receptor from human can be found in EP 1170365 as well as WO 00/32766. Like the other family members TRPV4 channel receptor is a Ca2+ permeable, non-selective, ligand-gated cation channel, which responds to diverse stimuli such as reduced osmolality, elevated temperature, and small molecule ligands. See, for instance, Voets, et al., J. Biol. Chem. (2002) 277 33704-47051; Watanabe, et al., J. Biol. Chem. (2002) 277:47044-47051; Watanabe, et al., J. Biol. Chem. (2002) 277: 13569-47051; Xu, et al., J. Biol. Chem. (2003) 278:11520-11527. From a screen of body tissues, the human TRPV4 channel receptor is most prominently expressed in cartilage. A screen of primary and clonal cell cultures shows significant expression only in chondrocytes.

In response to injurious compression and/or exposure to inflammatory mediators (e.g. inflammatory cytokines) chondrocytes decrease matrix production and increase production of multiple matrix degrading enzymes. Examples of matrix degrading enzymes include aggrecanases (ADAMTSs) and matrix metalloproteases (MMPs). The activities of these enzymes results in the degradation of the cartilage matrix. Aggrecanases (ADAMTSs), in conjunction with MMPs, degrade aggrecan, an aggregating proteoglycan present in articular cartilage. In osteoarthritic (OA) articular cartilage, a loss of proteoglycan staining is observed in the superficial zone in early OA and adjacent to areas of cartilage erosion in moderate to severe OA. The reduction in proteoglycan content is associated with an increase in degradation of type II collagen by specialized MMPs, termed collagenases (e.g. MMP-13). Collagenases are believed to make the initial cleavage within the triple-helix of intact collagen. It's hypothesized that the initial cleavage of collagen by collagenases facilitates the further degradation of the collagen fibrils by other proteases. Thus, preventing or reducing the increased production of matrix degrading enzymes and/or attenuating the inhibition of matrix production may also promote functional recovery. Modulation of TRPV4 channel receptor has been shown to play a role in attenuation of cartilage breakdown as well as a reduction or attenuation in the production of matrix degrading enzymes. See PCT/US2005/031872.

Excessive degradation of extracellular matrix is implicated in the pathogenesis of many diseases, including pain, chronic pain, neuropathic pain, postoperative pain, rheumatoid arthritis, osteoarthritis, neuralgia, neuropathies, algesia, nerve injury, ischaemia, neurodegeneration, cartilage degeneration, stroke, incontinence, inflammatory disorders, irritable bowel syndrome, obesity, periodontal disease, aberrant angiogenesis, tumor invasion and metastasis, corneal ulceration, and in complications of diabetes.

Thus, there is a need to discover new compounds useful in modulating TRPV4 channel receptors.

### SUMMARY OF THE INVENTION

This invention comprises compounds of the formula (I), as described hereinafter, which are useful in the treatment of diseases associated with TRPV4 channel receptors. This invention is also a pharmaceutical composition comprising a compound according to formula (I) and a pharmaceutically acceptable carrier. This invention is also a method of treating diseases associated with TRPV4 channel receptor in mammals, particularly in humans.

Specifically, the invention is directed to compounds according to Formula I

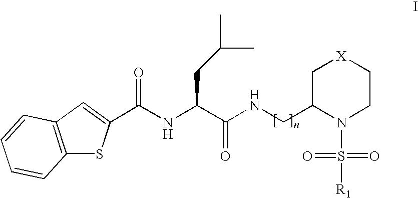

wherein
R¹ is optionally substituted aryl;

### X is CH₂, S, or SO₂, and

n=1 or 2.

### DETAILED DESCRIPTION OF THE INVENTION

In describing the invention, chemical elements are identified in accordance with the Periodic Table of the Elements. Abbreviations and symbols utilized herein are in accordance with the common usage of such abbreviations and symbols by those skilled in the chemical arts. For example, certain radical groups are abbreviated herein as follows: “t-Bu” refers to the tertiary butyl radical, “Boc” refers to the t-butyloxycarbonyl radical, “Fmoc” refers to the fluorenylmethoxycarbonyl radical, “Ph” refers to the phenyl radical, and “Cbz” refers to the benzyloxycarbonyl radical. In addition, certain reagents are abbreviated herein as follows: “m-CPBA” means 3-chloroperoxybenzoic acid, “EDC” means N-ethyl-N′(dimethylaminopropyl)-carbodiimide, “DMF” means dimethyl formamide, “DMSO” means dimethyl sulfoxide, “TEA” means triethylamine, “TFA” means trifluoroacetic acid, and “THF” means tetrahydrofuran.

### TERMS AND DEFINITIONS

The term “C₁-C₆ alkyl” as used herein at all occurrences means an optionally substituted, straight or branched chain radical of 1 to 6 carbon atoms, unless the chain length is limited thereto (e.g., C₁-C₄ means a radical of 1 to 4 carbon atoms), including, but not limited to methyl, ethyl, n-propyl, isopropyl, n-butyl, isobutyl and t-butyl, pentyl, n-pentyl, isopentyl, neopentyl and hexyl and isomers thereof.

“Amino acid” refers to the D- or L-isomers of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine and valine.

“Aryl” or “Ar” means optionally substituted phenyl or naphthyl.

“Enantiomerically enriched” refers to products whose enantiomeric excess is greater than zero. For example, enantiomerically enriched refers to products whose enantiomeric excess is greater than about 50% ee, greater than about 75% ee, and greater than about 90% ee.

“Enantiomeric excess” or “ee” is the excess of one enantiomer over the other expressed as a percentage. As a result, since both enantiomers are present in equal amounts in a racemic mixture, the enantiomeric excess is zero (0% ee). However, if one enantiomer was enriched such that it constitutes 95% of the product, then the enantiomeric excess would be 90% ee (the amount of the enriched enantiomer, 95%, minus the amount of the other enantiomer, 5%).

“Enantiomerically pure” refers to products whose enantiomeric excess is 100% ee.

“Diasteriomer” refers to a compound having at least two chiral centers.

“Diasteriomer excess” or “de” is the excess of one diasteriomer over the others expressed as a percentage.

“Diasteriomerically pure” refers to products whose diasteriomeric excess is 100% de.

“Half-life” (or “half-lives”) refers to the time required for half of a quantity of a substance to be converted to another chemically distinct species in vitro or in vivo.

“Halo” or “halogen” refers to fluoro, chloro, bromo, or iodo.

“Haloalkyl moieties” include 1-3 halogen atoms.

“Heteroatom” refers to a nitrogen, sulphur, or oxygen atom.

“Member atoms” refers to the atom or atoms that form a chain or ring. Where more than one member atom is present in a chain and within a ring, each member atom is covalently bound to an adjacent member atom in the chain or ring. Atoms that make up a substituent group on a chain or ring are not member atoms in the chain or ring.

“Optionally substituted” indicates that a group, such as alkyl, alkenyl, alkynyl, aryl, cycloalkyl, cycloalkenyl, heterocycloalkyl, heteroaryl, or phenyl may be substituted with one to three substituents as defined herein. “Optionally substituted” in reference to a group includes the unsubstituted group (e.g. “optionally substituted C₁-C₄alkyl” includes unsubstituted C₁-C₄alkyl). It should be understood that the term “substituted” includes the implicit provision that such substitution be in accordance with the permitted valence of the substituted atom and the substituent and that the substitution results in a stable compound (i.e. one that does not spontaneously undergo transformation such as by rearrangement, or cyclization). A single atom may be substituted with more than one substituent as long as such substitution is in accordance with the permitted valence of the atom. Suitable substituents include —OR, —C(O)R, —C(O)OR, —CH(R)OR, —SR, —S(O)R, —S(O)₂R, —N(R)(R), —N(R)C(O)OR, —N(R)C(O)R, —OC(O)N(R)(R), —N(H)C(═NR)N(R)(R)—C(O)N(R)(R), C(R)═NR, aryl, cyano, cycloalkyl, cycloalkenyl, halo, heterocycloalkyl, heteroaryl, nitro, and oxo; wherein each R is independently selected from H, alkyl, alkenyl, alkynyl, cycloalkyl, cycloalkenyl, aryl, heterocycloalkyl, and heteroaryl.

“Oxo” refers to the substituent group ═O.

As used herein, the term “physiologically functional derivative” refers to any pharmaceutically acceptable derivative of a compound of the present invention, for example, an ester or an amide, which upon administration to a mammal is capable of providing (directly or indirectly) a compound of the present invention or an active metabolite thereof. Such derivatives are clear to those skilled in the art, without undue experimentation, and with reference to the teaching of Burger's Medicinal Chemistry And Drug Discovery, 5th Edition, Vol 1: Principles and Practice, which is incorporated herein by reference to the extent that it teaches physiologically functional derivatives.

“Pharmaceutically acceptable” refers to those compounds, materials, compositions, and dosage forms which are, within the scope of sound medical judgment, suitable for use in contact with the tissues of human beings and animals without excessive toxicity, irritation, or other problem or complication, commensurate with a reasonable benefit/risk ratio.

Compounds within the invention may occur in two or more tautometric forms; all such tautomeric forms are included within the scope of the invention.

The term “Ph” represents a phenyl ring.

As used herein “agonist” to a TRPV4 channel receptor includes any compound capable of activating or enhancing the biological activities of a TRPV4 channel receptor.

As used herein “activating” the TRPV4 channel receptor may include, but is not limited to, such outcomes as increasing the amount of Ca²⁺ influx into a cell comprising a TRPV4 channel receptor, reducing the amount of ADAMTSs produced and/or released by the cell, reducing the amount of MMPs produced and/or released by the cell, inhibiting the basal or growth factor-stimulated proliferation of the cell, reducing the amount of nitric oxide (NO) produced by a cell, and attenuating the inhibition of matrix synthesis.

As used herein “inflammatory mediators” include any compound capable of triggering an inflammatory process. The term inflammation generally refers to the process of reaction of vascularized living tissue to injury. This process includes but is not limited to increased blood flow, increased vascular permeability, and leukocytic exudation. Because leukocytes recruited into inflammatory reactions can release potent enzymes and oxygen free radicals (i.e. inflammatory mediators), the inflammatory response is capable of mediating considerable tissue damage. Examples of inflammatory mediators include, but are not limited to prostaglandins (e.g. PGE2), leukotrienes (e.g. LTB4), inflammatory cytokines, such as tumour necrosis factor alpha (TNFα), interleukin 1 (IL-1), and interleukin 6 (IL-6); nitric oxide (NO), metalloproteinases, and heat shock proteins.

As used herein “matrix protein” includes proteins released from cells to form the extracellular matrix of cartilage. The extracellular matrix of cartilage consists of proteoglycans, belonging to several distinct proteoglycan families. These include, but are not limited to, perlecan and the hyalectans, exemplified by aggrecan and versican, and the small leucine-rich family of proteoglycans, including decorin, biglycan and fibromodulin. The extracellular matrix also consists of hybrid collagen fibers comprised of three collagen isotypes, namely type II, type IX, and type XI collagens, along with accessory proteins such as cartilage oligeromeric matrix protein (COMP), link protein, and fibronectin.

Cartilage also contains hyaluronin which forms a noncovalent association with the hyalectins. In addition, a specialized pericellular matrix surrounds the chondrocyte which consists of proteoglycans, type VI collagen and collagen receptor proteins, such as anchorin.

As used herein “matrix degrading enzymes” refers to enzymes able to cleave extracellular matrix proteins. Cartilage extracellular matrix turnover is regulated by matrix metalloproteases (MMPs) which are synthesized as latent proenzymes that require activation in order to degrade cartilage extracellular matrix proteins. Three classes of enzymes are believed to regulate the turnover of extracellular matrix proteins, namely collagenases (including, but not limited to, MMP-13), responsible for the degradation of native collagen fibers, stromelysins (including, but not limited to, MMP-3) which degrade proteoglycan and type IX collagen, and gelatinases (including, but not limited to, MMP-2 and MMP-9) which degrade denatured collagen. The matrix degrading enzyme group that appears most relevant in cartilage degradation in OA includes a subgroup of metalloproteinases called ADAMTS, because they possess disintegrin and metalloproteinase domains and a thrombospondin motif in their structure. ADAMTS4 (aggrecanase-1) has been reported to be elevated in OA joints and along with ADAMTS-5 (aggrecanase-2) have been shown to be expressed in human osteoarthritic cartilage. These enzymes appear to be responsible for aggrecan degradation without MMP participation. Thus, an inhibition of activity or a reduction in expression of these enzymes may have utility in OA therapy.

As used herein, “reduce” or “reducing” the production of matrix degrading enzymes refers to a decrease in the amount of matrix degrading enzyme(s) produced and/or released by a cell, which has exhibited an increase in matrix degrading enzyme production or release in response to a catabolic stimulus, which may include, but is not limited to, physical injury, mechanical and/or osmotic stress, or exposure to an inflammatory mediator.

As used herein “attenuate” or “attenuating” refers to a normalization (i.e., either an increase or decrease) of the amount of matrix degrading enzyme, inflammatory mediator, or matrix protein produced and/or released by a cell, following exposure to a catabolic stimulus. For example, following exposure to IL-1 chondrocyte production of matrix proteins, such as proteoglycans, are reduced, while production of matrix degrading enzymes (e.g. MMP-13, ADAMTS4) and reactive oxygen species (e.g. NO) are increased. Attenuation refers to the normalization of these diverse responses to levels observed in the absence of a catabolic stimulus.

Some of the compounds of this invention may be crystallised or recrystallised from solvents such as aqueous and organic solvents. In such cases solvates may be formed. This invention includes within its scope stoichiometric solvates including hydrates as well as compounds containing variable amounts of water that may be produced by processes such as lyophilisation.

Since the compounds of formula (I) are intended for use in pharmaceutical compositions it will readily be understood that they are each provided in substantially pure form, for example at least 60% pure, more suitably at least 75% pure or at least 85%, especially at least 98% pure (% are on a weight for weight basis). Impure preparations of the compounds may be used for preparing the more pure forms used in the pharmaceutical compositions; these less pure preparations of the compounds should contain at least 1%, more suitably at least 5% or from 10 to 59% of a compound of the formula (I) or pharmaceutically acceptable derivative thereof.

Pharmaceutically acceptable salts of the compounds of Formula (I) are readily prepared by those of skill in the art. Compounds of formula (I) may also be prepared as the N-oxide. Compounds of formula (I) having a free carboxy group may also be prepared as an in vivo hydrolysable ester. The invention extends to all such derivatives.

Certain of the above-mentioned compounds of formula (I) may exist in the form of optical isomers, e.g. diastereoisomers and mixtures of isomers in all ratios, e.g. racemic mixtures. The invention includes all such forms, in particular the pure isomeric forms. The different isomeric forms may be separated or resolved one from the other by conventional methods, or any given isomer may be obtained by conventional synthetic methods or by stereospecific or asymmetric syntheses.

The composition may be formulated for administration by any route, such as oral, topical or parenteral. The compositions may be in the form of tablets, capsules, powders, granules, lozenges, creams or liquid preparations, such as oral or sterile parenteral solutions or suspensions.

The topical formulations of the present invention may be presented as, for instance, ointments, creams or lotions, eye ointments and eye or ear drops, impregnated dressings and aerosols, and may contain appropriate conventional additives such as preservatives, solvents to assist drug penetration and emollients in ointments and creams.

The formulations may also contain compatible conventional carriers, such as cream or ointment bases and ethanol or oleyl alcohol for lotions. Such carriers may be present as from about 1% up to about 98% of the formulation.

More usually they will form up to about 80% of the formulation. Tablets and capsules for oral administration may be in unit dose presentation form, and may contain conventional excipients such as binding agents, for example syrup, acacia, gelatin, sorbitol, tragacanth, or polyvinylpyrollidone; fillers, for example lactose, sugar, maize-starch, calcium phosphate, sorbitol or glycine; tabletting lubricants, for example magnesium stearate, talc, polyethylene glycol or silica; disintegrants, for example potato starch; or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatin, hydroxyethyl cellulose, carboxymethyl cellulose, aluminium stearate gel or hydrogenated edible fats, emulsifying agents, for example lecithin, sorbitan monooleate, or acacia; non-aqueous vehicles (which may include edible oils), for example almond oil, oily esters such as glycerine, propylene glycol, or ethyl alcohol; preservatives, for example methyl or propyl p-hydroxybenzoate or sorbic acid, and, if desired, conventional flavouring or colouring agents.

Suppositories will contain conventional suppository bases, e.g. cocoa-butter or other glyceride.

For parenteral administration, fluid unit dosage forms are prepared utilizing the compound and a sterile vehicle, water being preferred. The compound, depending on the vehicle and concentration used, can be either suspended or dissolved in the vehicle. In preparing solutions the compound can be dissolved in water for injection and filter sterilised before filling into a suitable vial or ampoule and sealing.

Advantageously, agents such as a local anaesthetic, preservative and buffering agents can be dissolved in the vehicle. To enhance the stability, the composition can be frozen after filling into the vial and the water removed under vacuum. The dry lyophilized powder is then sealed in the vial and an accompanying vial of water for injection may be supplied to reconstitute the liquid prior to use. Parenteral suspensions are prepared in substantially the same manner except that the compound is suspended in the vehicle instead of being dissolved and sterilization cannot be accomplished by filtration. The compound can be sterilised by exposure to ethylene oxide before suspending in the sterile vehicle. Advantageously, a surfactant or wetting agent is included in the composition to facilitate uniform distribution of the compound.

The invention is directed to compounds according to Formula I:

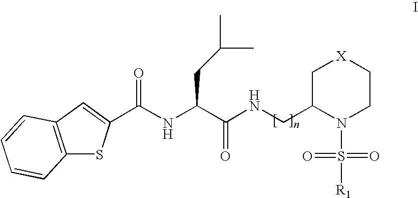

wherein
R¹ is optionally substituted aryl;

### X is CH₂, S, or SO₂, and

n=1 or 2.

In another aspect the present invention also includes, a pharmaceutical composition comprising a compound of Formula I and a pharmaceutically acceptable carrier, diluent or excipient.

The meaning of any functional group or substituent thereon at any one occurrence in Formula I, or any subformula thereof, is independent of its meaning, or any other functional group's or substituent's meaning, at any other occurrence, unless stated otherwise.

The compounds according to Formula I may contain one or more asymmetric centers and may, therefore, exist as individual enantiomers, diasteriomers, or other stereoisomeric forms, or as mixtures thereof. Asymmetric carbon atoms may be present in a substituent such as an alkyl group. Where the stereochemistry of chiral carbons present in Formula I, or in any chemical structure illustrated herein, is not specified, the chemical structure is intended to encompass compounds containing any stereoisomer and all mixtures thereof of each chiral center present in the compound. Thus, compounds according to Formula I containing one or more chiral centers may be used as racemic mixtures, enantiomerically enriched mixtures, or as enantiomerically pure individual stereoisomers.

Individual stereoisomers of a compound according to Formula I which contain one or more asymmetric centers may be resolved by methods known to those skilled in the art. For example, such resolution may be carried out by formation of diastereoisomeric salts or complexes which may be separated, for example, by crystallisation; by formation of diastereoisomeric derivatives which may be separated, for example, by crystallisation, gas-liquid or liquid chromatography; by selective reaction of one enantiomer with an enantiomer-specific reagent, for example by enzamatic oxidation or reduction, followed by separation of the modified and unmodified enantiomers; or gas-liquid or liquid chromatography in a chiral environment, for example, on a chiral support such as silica with a bound chiral ligand or in the presence of a chiral solvent. The skilled artisan will appreciate that where the desired enantiomer is converted into another chemical entity by one of the separation procedures described above, a further step is required to liberate the desired enantiomeric form. Alternatively, specific enantiomers may be synthesized by asymmetric synthesis using optically active reagents, substrates, catalysts or solvents, or by converting one enantiomer to the other by asymmetric transformation.

The compounds according to Formula I may also contain double bonds or other centers of geometric asymmetry. Formula I includes both trans (E) and cis (Z) geometric isomers. Likewise, all tautomeric forms are also included in Formula I whether such tautomers exist in equilibrium or predominately in one form.

The skilled artisan will appreciate that pharmaceutically-acceptable salts of the compounds according to Formula I can be prepared. Indeed, in certain embodiments of the invention, pharmaceutically-acceptable salts of the compounds according to Formula I may be preferred over the respective free base or free acid because such salts impart greater stability or solubility to the molecule thereby facilitating formulation into a dosage form. Accordingly, the invention is further directed to pharmaceutically-acceptable salts of the compounds according to Formula I.

As used herein, the term “pharmaceutically-acceptable salts” refers to salts that retain the desired biological activity of the subject compound and exhibit minimal undesired toxicological effects. The term “pharmaceutically-acceptable salts” includes both pharmaceutically-acceptable acid addition salts and pharmaceutically-acceptable base addition salts. These pharmaceutically-acceptable salts may be prepared in situ during the final isolation and purification of the compound, or by separately reacting the purified compound in its free acid or free base form with a suitable base or acid, respectively.

In certain embodiments, compounds according to Formula I may contain an acidic functional group and are therefore capable of forming pharmaceutically-acceptable base addition salts by treatment with a suitable base. Suitable bases include ammonia and hydroxides, carbonates and bicarbonates of a pharmaceutically-acceptable metal cation, such as alkali metal and alkaline earth metal cations. Suitable alkali metal and alkaline earth metal cations include sodium, potassium, lithium, calcium, magnesium, aluminum, and zinc. Suitable bases further include pharmaceutically-acceptable organic primary, secondary, and tertiary amines including aliphatic amines, aromatic amines, aliphatic diamines, and hydroxy alkylamines. Suitable pharmaceutically-acceptable organic bases include methylamine, ethylamine, diethylamine, ethylenediamine, ethanolamine, diethanolamine, and cyclohexylamine.

In certain embodiments, compounds according to Formula I may contain a basic functional group and are therefore capable of forming pharmaceutically-acceptable acid addition salts by treatment with a suitable acid. Suitable acids include, but are not limited to, pharmaceutically-acceptable inorganic acids, pharmaceutically-acceptable organic acids, and pharmaceutically-acceptable organic sulfonic acids. Suitable inorganic acids include, but are not limited to, hydrochloric acid, hydrobromic acid, nitric acid, sulfuric acid, sulfamic acid, and phosphoric acid. Suitable organic acids include, acetic acid, hydroxyacetic acid, propionic acid, butyric acid, isobutyric acid, maleic acid, hydroxymaleic acid, acrylic acid, fumaric acid, malic acid, tartaric acid, citric acid, salicylic acid, p-aminosalicyclic acid, glycollic acid, lactic acid, heptanoic acid, phthalic acid, oxalic acid, succinic acid, benzoic acid, o-acetoxybenzoic acid, chlorobenzoic acid, methylbenzoic acid, dinitrobenzoic acid, hydroxybenzoic acid, methoxybenzoic acid, phenylacetic acid, mandelic acid, formic acid, stearic acid, ascorbic acid, palmitic acid, oleic acid, pyruvic acid, pamoic acid, malonic acid, lauric acid, glutaric acid, and glutamic acid. Suitable organic sulfonic acids include, methanesulfonic acid, ethanesulfonic acid, 2-hydroxyethanesulfonic acid, benzenesulfonic acid, p-aminobenzenesulfonic (i.e. sulfanilic acid), p-toluenesulfonic acid, and napthalene-2-sulfonic acid.

As used herein, the term “compounds of the invention” means both the compounds according to Formula I and the pharmaceutically-acceptable salts thereof. The term “a compound of the invention” also appears herein and refers to both a compound according to Formula I and its pharmaceutically-acceptable salts.

The compounds of the invention may exist as solids, liquids, or gases, all of which are included in the invention. In the solid state, the compounds of the invention may exist as either amorphous material or in crystalline form, or as a mixture thereof. The skilled artisan will appreciate that pharmaceutically-acceptable solvates of the compounds of the invention may be formed wherein solvent molecules are incorporated into the crystalline lattice during crystallization. Solvates may involve nonaqueous solvents such as ethanol, isopropanol, DMSO, acetic acid, ethanolamine, and ethyl acetate, or they may involve water as the solvent that is incorporated into the crystalline lattice. Solvates wherein water is the solvent that is incorporated into the crystalline lattice are typically referred to as “hydrates.” The invention includes all such solvates.

The skilled artisan will further appreciate that certain compounds of the invention that exist in crystalline form, including the various solvates thereof, may exhibit polymorphism (i.e. the capacity to occur in different crystalline structures). These different crystalline forms are typically known as “polymorphs.” The invention includes all such polymorphs. Polymorphs have the same chemical composition but differ in packing, geometrical arrangement, and other descriptive properties of the crystalline solid state. Polymorphs, therefore, may have different physical properties such as shape, density, hardness, deformability, stability, and dissolution properties. Polymorphs typically exhibit different melting points, IR spectra, and X-ray powder diffraction patterns, which may be used for identification. The skilled artisan will appreciate that different polymorphs may be produced, for example, by changing or adjusting the reaction conditions or reagents, such as solvents, used in making the compound. In addition, one polymorph may spontaneously convert to another polymorph under certain conditions.

In another aspect of the invention, R¹ is optionally substituted phenyl. The optionally substituted phenyl may be substituted with one to three of CN, NO₂, or halogen.

Exemplary compounds of this invention include:
N-((1S)-1-{[({1-[(2-cyanophenyl)sulfonyl]-2-piperidinyl}methyl)amino]carbonyl}-3-methylbutyl)-1-benzothiophene-2-carboxamide;
N-((1S)-1-{[(2-{1-[(2-chloro-4-fluorophenyl)sulfonyl]-2-piperidinyl}ethyl)amino]carbonyl}-3-methylbutyl)-1-benzothiophene-2-carboxamide;
N-((1S)-1-{[(2-{1-[(2,4-dichlorophenyl)sulfonyl]-2-piperidinyl}ethyl)amino]carbonyl}-3-methylbutyl)-1-benzothiophene-2-carboxamide;
N-((1S)-1-{[(2-{4-[(2,4-dichlorophenyl)sulfonyl]-3-thiomorpholinyl}ethyl)amino]carbonyl}-3-methylbutyl)-1-benzothiophene-2-carboxamide; and
N-((1S)-1-{[(2-{4-[(2,4-dichlorophenyl)sulfonyl]-1,1-dioxido-3-thiomorpholinyl}ethyl)amino]carbonyl}-3-methylbutyl)-1-benzothiophene-2-carboxamide.

### Synthetic Schemes:

The synthesis of the compounds of the general formula (I) may be accomplished as outlined below in Schemes 1-3.

Scheme 1 outlines the assembly of 2-methylaminopiperidine analogs. Starting from commercially available starting material 1, coupling with an amino acid such as, but not limited to, CBZ-leucine is accomplished under conditions common to the art such as EDC and HOBt to generate amide 2. Hydrogenolysis under standard conditions in the presence of palladium on carbon and hydrogen atmosphere cleanly removed the CBZ protecting group to provide amine 3. Another peptide coupling reaction facilitated by EDC and HOBt in the presence of a carboxylic acid such as, but not limited to, benzothiophene-2-carboxylate and a base such as triethylamine leads to intermediate 4 which undergoes subsequent treatment with an acid such as hydrochloric acid in the presence of methanol to remove the tert-butyl carbonyl group giving amine 5. Treatment with an electrophilic reagent such as, but not limited to, 2-cyanobenzenesulfonyl chloride in the presence of an amine base such as triethylamine provides the target compound 6 as a mixture of diastereomers.

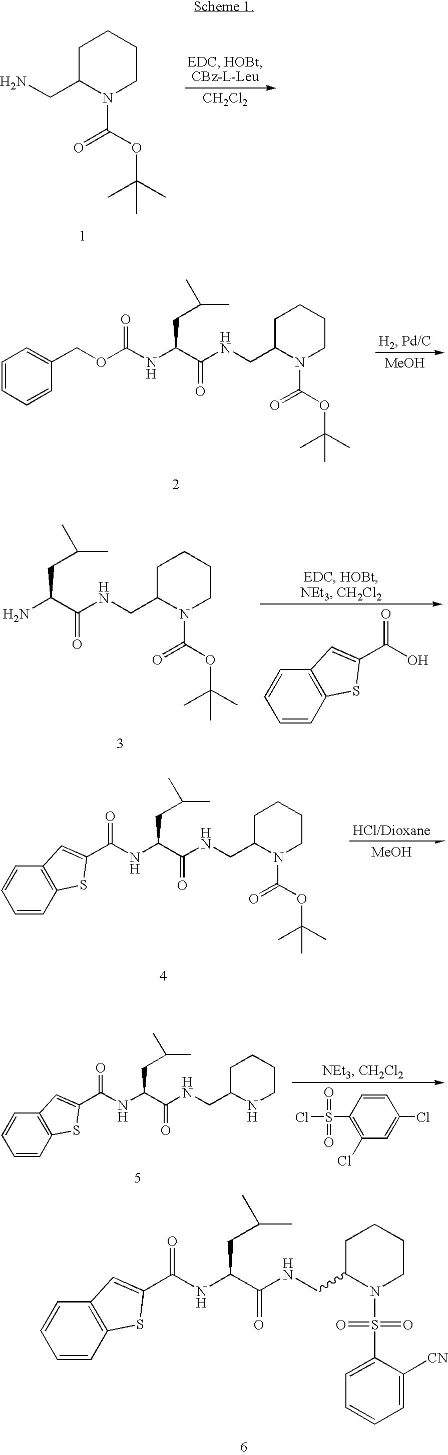

Scheme 2 details the preparation of 2-ethylaminopiperidine analogs. Starting from commercially available starting material 7, coupling with benzothiophene-leucine peptide is accomplished under conditions common to the art utilizing reagents such as EDC and HOBt in the presence of an amine base such as triethylamine to provide the intermediate 8. Subsequent removal of the Boc protecting group under standard conditions such as hydrochloric acid in methanol and treatment with an electrophilic reagent such as 2,4-dichlorophenylsulfonyl chloride or 2-chloro-4-fluorophenylsulfonyl chloride in the presence of an amine base such as triethylamine provides the target compounds 10 or 11 as a diastereomeric mixture.

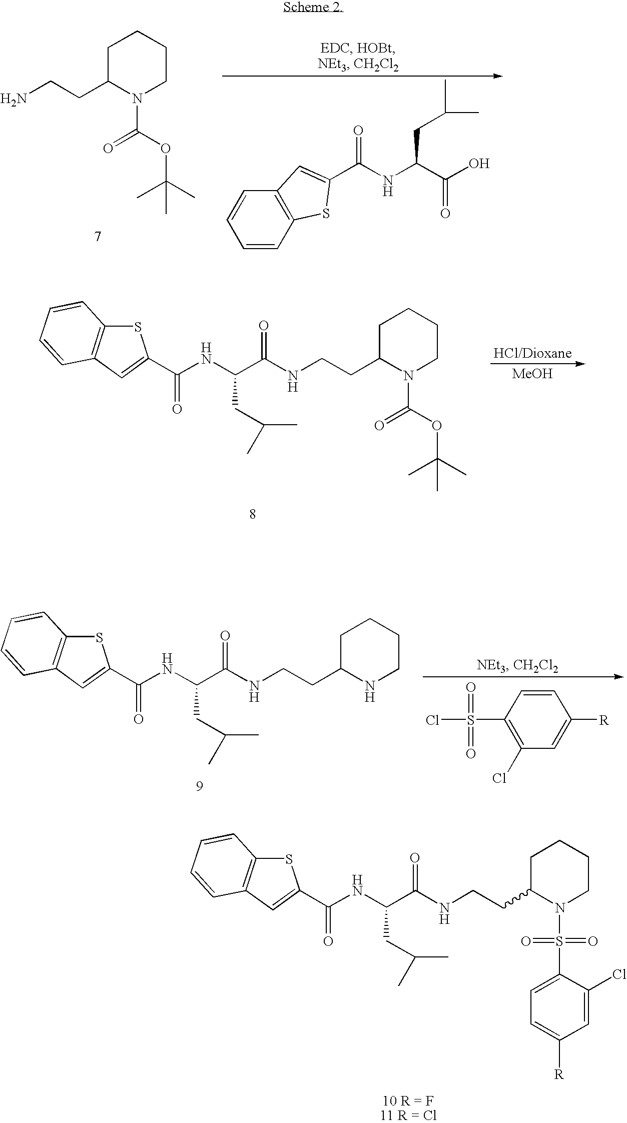

The preparation of other analogs is delineated in Scheme 3. Beginning with commercially available starting material, the carboxylic acid 12 is converted to a methyl ester and then reduced to the alcohol under standard conditions common to the art. By a two-step process, the alcohol is converted to a methylsulfonate group under standard conditions using methanesulfonyl chlorided in the presence of an amine base such as triethylamine and then the methylsulfonate is displaced by treatment with potassium cyanide in a polar aprotic solvent such as dimethylsulfoxide to generate the nitrile 14. Hydrogenation of the nitrile under standard conditions using a metal catalyst such as Raney nickel under a hydrogen atmosphere provided the amine 15, which was subsequently coupled with the benzothiophene-leucine acid under standard peptide coupling conditions to provide the amine intermediate 16. Removal of the tert-butyl carbonyl protecting group under conditions common to the art such as hydrochloric acid in methanol and subsequent treatment of the secondary amine with an electrophilic reagent such as 2,4-dichlorophenylsulfonyl chloride provides the target compound 18. The thiomorpholine can be further modified by treatment with meta-chloroperbenzoic acid under standard conditions to generate sulfone 19.

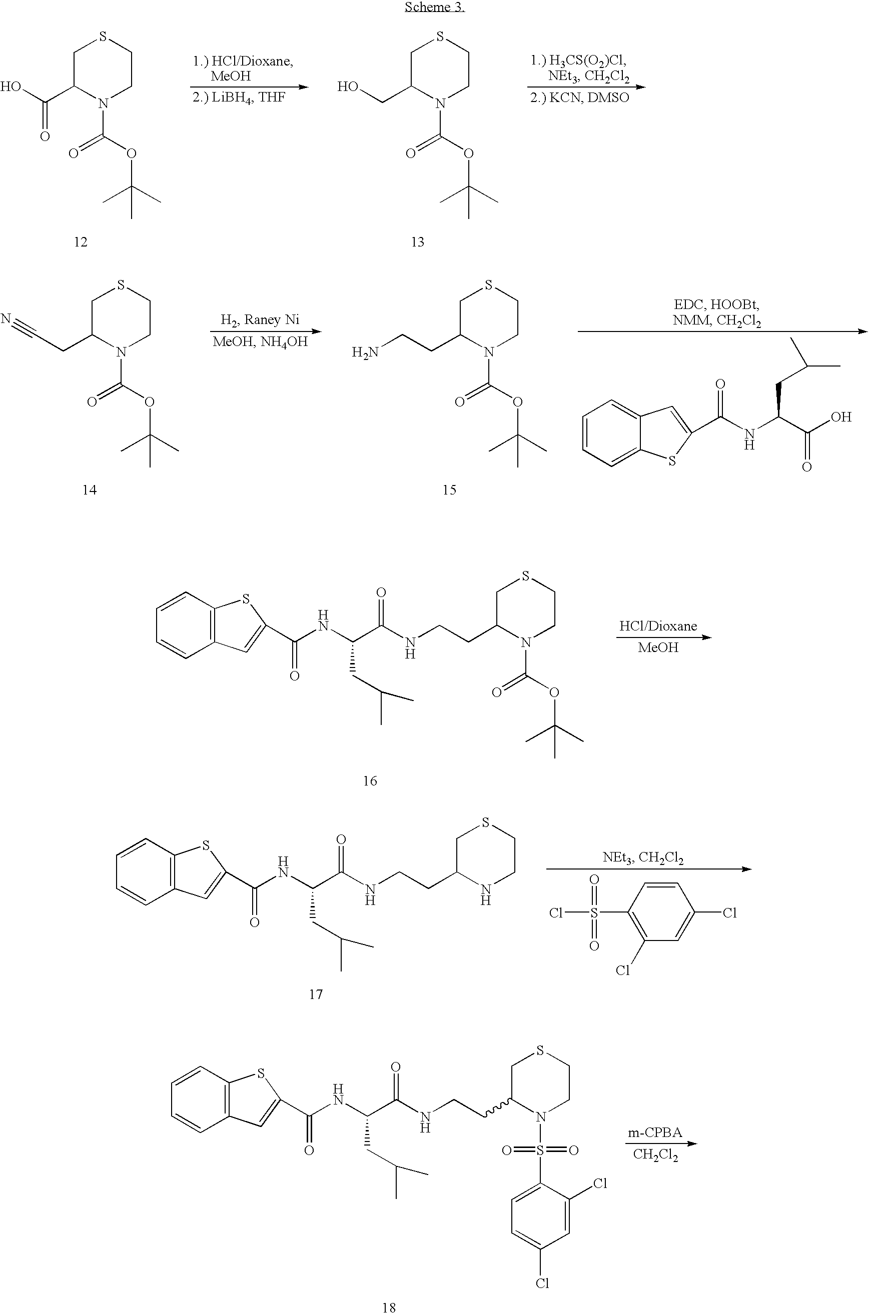

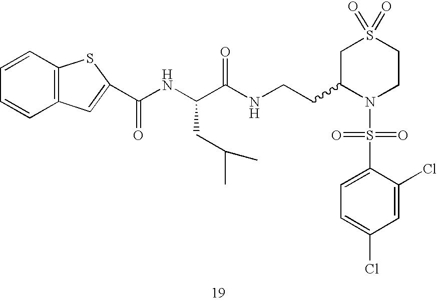

### Compositions

The compounds of the invention will normally, but not necessarily, be formulated into pharmaceutical compositions prior to administration to a patient. Accordingly, in another aspect the invention is directed to pharmaceutical compositions comprising a compound of the invention and a pharmaceutically-acceptable excipient.

The pharmaceutical compositions of the invention may be prepared and packaged in bulk form wherein a safe and effective amount of a compound of the invention can be extracted and then given to the patient such as with powders or syrups. Alternatively, the pharmaceutical compositions of the invention may be prepared and packaged in unit dosage form wherein each physically discrete unit contains a safe and effective amount of a compound of the invention. When prepared in unit dosage form, the pharmaceutical compositions of the invention typically contain from about 0.1 mg to about 50 mg.

The pharmaceutical compositions of the invention typically contain one compound of the invention. However, in certain embodiments, the pharmaceutical compositions of the invention contain more than one compound of the invention. For example, in certain embodiments the pharmaceutical compositions of the invention contain two compounds of the invention. In addition, the pharmaceutical compositions of the invention may optionally further comprise one or more additional pharmaceutically active compounds. Conversely, the pharmaceutical compositions of the invention typically contain more than one pharmaceutically-acceptable excipient. However, in certain embodiments, the pharmaceutical compositions of the invention contain one pharmaceutically-acceptable excipient.

As used herein, “pharmaceutically-acceptable excipient” means a pharmaceutically acceptable material, composition or vehicle involved in giving form or consistency to the pharmaceutical composition. Each excipient must be compatible with the other ingredients of the pharmaceutical composition when commingled such that interactions which would substantially reduce the efficacy of the compound of the invention when administered to a patient and interactions which would result in pharmaceutical compositions that are not pharmaceutically acceptable are avoided. In addition, each excipient must of course be of sufficiently high purity to render it pharmaceutically-acceptable.

The compound of the invention and the pharmaceutically-acceptable excipient or excipients will typically be formulated into a dosage form adapted for administration to the patient by the desired route of administration. For example, dosage forms include those adapted for (1) oral administration such as tablets, capsules, caplets, pills, troches, powders, syrups, elixers, suspensions, solutions, emulsions, sachets, and cachets; (2) parenteral administration such as sterile solutions, suspensions, and powders for reconstitution; (3) transdermal administration such as transdermal patches; (4) rectal administration such as suppositories; (5) inhalation such as aerosols and solutions; and (6) topical administration such as creams, ointments, lotions, solutions, pastes, sprays, foams, and gels.

Suitable pharmaceutically-acceptable excipients will vary depending upon the particular dosage form chosen. In addition, suitable pharmaceutically-acceptable excipients may be chosen for a particular function that they may serve in the composition. For example, certain pharmaceutically-acceptable excipients may be chosen for their ability to facilitate the production of uniform dosage forms. Certain pharmaceutically-acceptable excipients may be chosen for their ability to facilitate the production of stable dosage forms. Certain pharmaceutically-acceptable excipients may be chosen for their ability to facilitate the carrying or transporting the compound or compounds of the invention once administered to the patient from one organ, or portion of the body, to another organ, or portion of the body. Certain pharmaceutically-acceptable excipients may be chosen for their ability to enhance patient compliance.

Suitable pharmaceutically-acceptable excipients include, but are not limited to, the following types of excipients: diluents, fillers, binders, disintegrants, lubricants, glidants, granulating agents, coating agents, wetting agents, solvents, co-solvents, suspending agents, emulsifiers, sweeteners, flavoring agents, flavor masking agents, coloring agents, anticaking agents, hemectants, chelating agents, plasticizers, viscosity increasing agents, antioxidants, preservatives, stabilizers, surfactants, and buffering agents. The skilled artisan will appreciate that certain pharmaceutically-acceptable excipients may serve more than one function and may serve alternative functions depending on how much of the excipient is present in the formulation and what other ingredients are present in the formulation.

Skilled artisans possess the knowledge and skill in the art to enable them to select suitable pharmaceutically-acceptable excipients in appropriate amounts for use in the invention. In addition, there are a number of resources that are available to the skilled artisan which describe pharmaceutically-acceptable excipients and may be useful in selecting suitable pharmaceutically-acceptable excipients. Examples include Remington's Pharmaceutical Sciences (Mack Publishing Company), The Handbook of Pharmaceutical Additives (Gower Publishing Limited), and The Handbook of Pharmaceutical Excipients (the American Pharmaceutical Association and the Pharmaceutical Press).

The pharmaceutical compositions of the invention are prepared using techniques and methods known to those skilled in the art. Some of the methods commonly used in the art are described in Remington's Pharmaceutical Sciences (Mack Publishing Company).

In one aspect, the invention is directed to a solid oral dosage form such as a tablet or capsule comprising a safe and effective amount of a compound of the invention and a diluent or filler. Suitable diluents and fillers include lactose, sucrose, dextrose, mannitol, sorbitol, starch (e.g. corn starch, potato starch, and pre-gelatinized starch), cellulose and its derivatives (e.g. microcrystalline cellulose), calcium sulfate, and dibasic calcium phosphate. The oral solid dosage form may further comprise a binder. Suitable binders include starch (e.g. corn starch, potato starch, and pre-gelatinized starch), gelatin, acacia, sodium alginate, alginic acid, tragacanth, guar gum, povidone, and cellulose and its derivatives (e.g. microcrystalline cellulose). The oral solid dosage form may further comprise a disintegrant. Suitable disintegrants include crospovidone, sodium starch glycolate, croscarmelose, alginic acid, and sodium carboxymethyl cellulose. The oral solid dosage form may further comprise a lubricant. Suitable lubricants include stearic acid, magnesium stearate, calcium stearate, and talc.

### Biological Assays

The compounds of this invention may be tested in one of several biological assays.

Ca²⁺ influx mediated through TRPV4 channel receptors can be measured using articular chondrocytes from such species as, but not limited to, human, rat, canine, rabbit, monkey, and bovine, using standard techniques in the art such as, but not limited to, Fura-2 (Invitrogen/Molecular Probes, Eugene, Oreg.) fluorescence using a FlexStation (manufactured by Molecular Devices, Sunnyvale, Calif.). Table 1 lists biological data for several representative compounds obtained using this method in bovine articular chondrocytes.

TABLE 1
Ca²⁺ influx in bovine articular chondrocytes

| Compound Example No. | EC50 values |
|---|---|
| 3 | +++ |
| 1 | ++ |

Legend

| EC₅₀ values | |
|---|---|
| (in micromolar) | Symbol |
| 0.01-0.10 | +++ |
| 0.11-2.0 | ++ |

Other techniques used to measure TRPV4 channel receptor activation in chondrocytes include, but are not limited to: FLIPR assay, measuring a compound's capability to reduce the amount of ADAMTSs produced and/or released in response to a catabolic stimulus by a cell comprising a TRPV4 channel receptor; measuring a compound's capability to reduce the amount of MMPs produced and/or released in response to a catabolic stimulus by a cell comprising a TRPV4 channel receptor; measuring a compound's capability to effect the amount of nitric oxide (NO) produced in response to a catabolic stimulus by a cell comprising a TRPV4 channel receptor; and measuring a compound's capability to attenuate the inhibition of matrix synthesis in response to a catabolic stimulus by a cell comprising a TRPV4 channel receptor.

The compounds of this invention generally show TRPV4 channel receptor modulator activity having EC50 values in the range of 0.01 μM to 10 μM. The full structure/activity relationship has not yet been established for the compounds of this invention; nevertheless, one of ordinary skill in the art can readily determine which compounds of formula (I) are modulators of the TRPV4 channel receptor with an EC₅₀ value advantageously in the range of 0.01 μM to 10 μM using an assay described herein. All exemplary compounds of the present invention were assessed using at least one of the biological assays presented above. Compounds presented in the Examples had EC₅₀ values of about 0.01 μM to 10 μM as measured by Flex Station using bovine and/or human articular chondrocytes.

### Methods of Use

The compounds of the present invention are agonists of TRPV4 channel receptors. The compounds of the present invention are useful in the treatment of disease associated with TRPV4 channel receptors. Thus, the present invention provides a method of activating a TRPV4 channel receptor in a patient, comprising administering to said patient in need thereof an effective amount of a compound of formula I. Also provided is a method for treating a patient in need thereof comprising contacting at least one cell expressing a TRPV4 channel receptor of the patient with a therapeutically effective amount of a compound of formula I.

In one aspect of the present invention, the patient suffers from a disease affecting cartilage or matrix degradation. In another aspect, the patient is suffering from a disease or condition chosen from the group of: pain, chronic pain, neuropathic pain, postoperative pain, rheumatoid arthritis, osteoarthritis, neuralgia, neuropathies, algesia, nerve injury, ischaemia, neurodegeneration, cartilage degeneration, and inflammatory disorders. In another aspect, the patient suffers from a disease affecting the larynx, trachea, auditory canal, intervertebral discs, ligaments, tendons, joint capsules or bone development. In another aspect the disease is osteoarthritis. In another aspect the disease is rheumatoid arthritis. The methods of treatment of the invention comprise administering a safe and effective amount of a compound according to Formula I or a pharmaceutically-acceptable salt thereof to a patient in need thereof.

As used herein, “treatment” means: (1) the amelioration or prevention of the condition being treated or one or more of the biological manifestations of the condition being treated, (2) the interference with (a) one or more points in the biological cascade that leads to or is responsible for the condition being treated or (b) one or more of the biological manifestations of the condition being treated, or (3) the alleviation of one or more of the symptoms or effects associated with the condition being treated. The skilled artisan will appreciate that “prevention” is not an absolute term. In medicine, “prevention” is understood to refer to the prophylactic administration of a drug to substantially diminish the likelihood or severity of a condition or biological manifestation thereof, or to delay the onset of such condition or biological manifestation thereof.

As used herein, “safe and effective amount” means an amount of the compound sufficient to significantly induce a positive modification in the condition to be treated but low enough to avoid serious side effects (at a reasonable benefit/risk ratio) within the scope of sound medical judgment. A safe and effective amount of a compound of the invention will vary with the particular compound chosen (e.g. consider the potency, efficacy, and half-life of the compound); the route of administration chosen; the condition being treated; the severity of the condition being treated; the age, size, weight, and physical condition of the patient being treated; the medical history of the patient to be treated; the duration of the treatment; the nature of concurrent therapy; the desired therapeutic effect; and like factors, but can nevertheless be routinely determined by the skilled artisan.

As used herein, “patient” refers to a human or other animal.

The compounds of the invention may be administered by any suitable route of administration, including both systemic administration and topical administration. Systemic administration includes oral administration, parenteral administration, transdermal administration, rectal administration, and administration by inhalation. Parenteral administration refers to routes of administration other than enteral, transdermal, or by inhalation, and is typically by injection or infusion. Parenteral administration includes intravenous, intramuscular, and subcutaneous injection or infusion. Inhalation refers to administration into the patient's lungs whether inhaled through the mouth or through the nasal passages. Topical administration includes application to the skin as well as intraocular, otic, intravaginal, and intranasal administration.

The compounds of the invention may be administered once or according to a dosing regimen wherein a number of doses are administered at varying intervals of time for a given period of time. For example, doses may be administered one, two, three, or four times per day. Doses may be administered until the desired therapeutic effect is achieved or indefinitely to maintain the desired therapeutic effect. Suitable dosing regimens for a compound of the invention depend on the pharmacokinetic properties of that compound, such as absorption, distribution, and half-life, which can be determined by the skilled artisan. In addition, suitable dosing regimens, including the duration such regimens are administered, for a compound of the invention depend on the condition being treated, the severity of the condition being treated, the age and physical condition of the patient being treated, the medical history of the patient to be treated, the nature of concurrent therapy, the desired therapeutic effect, and like factors within the knowledge and expertise of the skilled artisan. It will be further understood by such skilled artisans that suitable dosing regimens may require adjustment given an individual patient's response to the dosing regimen or over time as individual patient needs change.

Typical daily dosages may vary depending upon the particular route of administration chosen. Typical daily dosages for oral administration range from about 0.4 to about 400 mg/kg. Typical daily dosages for parenteral administration range from about 0.01 to about 100 mg/kg; or between 0.1 and 20 mg/kg. The compounds of the invention may be administered alone or in combination with one or more additional active agents.

### EXAMPLES

The following examples illustrate the invention. These examples are not intended to limit the scope of the invention, but rather to provide guidance to the skilled artisan to prepare and use the compounds, compositions, and methods of the invention. While particular embodiments of the invention are described, the skilled artisan will appreciate that various changes and modifications can be made without departing from the spirit and scope of the invention.

### Example 1

### Preparation of N-((1S)-1-{[({1-[(2-cyanophenyl)sulfonyl]-2-piperidinyl}methyl)amino]carbonyl}-3-methylbutyl)-1-benzothiophene-2-carboxamide

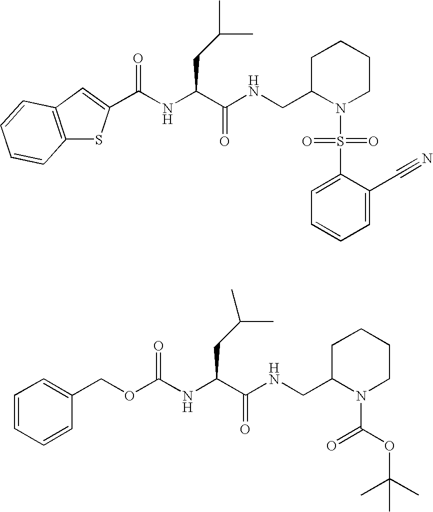

### a. 1,1-Dimethylethyl 2-{[(N-{[(phenylmethyl)oxy]carbonyl}-L-leucyl)amino]methyl}-1-piperidinecarboxylate

To a solution of 2-(aminomethyl)-1-N-boc-piperidine (0.610 g, 2.85 mmol) in CH₂Cl₂ (19 mL) was added HOBt (0.463 g, 3.43 mmol), Cbz-L-Leucine (0.819 g, 3.09 mmol), and EDC (0.654 g, 3.41 mmol). The reaction was stirred at room temperature for 20 h. The reaction mixture was diluted with CH₂Cl₂ and washed successively with 1N HCl, sat. NaHCO₃, and brine. The organic layer was dried over Na₂SO₄, filtered, and concentrated to afford 1.24 g of crude the title compound which was carried to the next step: LCMS (m/z) 462.2 (M+H)⁺.

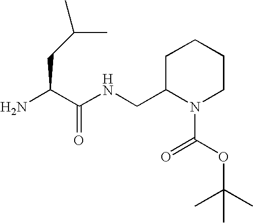

### b. 1,1-Dimethylethyl 2-[(L-leucylamino)methyl]-1-piperidinecarboxylate

To a purged (N₂) solution of the product from Example 1a (1.24 g, 2.69 mmol) in methanol (18 mL) was added 10% Pd/C (0.163 g). The reaction was stirred under balloon pressure of H₂ for 18 h and was then filtered through Celite. The solid was washed with CH₃OH and CH₂Cl₂, and the combined filtrate was concentrated to afford 0.940 g of the crude title compound: LCMS (m/z) 328.2 (M+H)⁺.

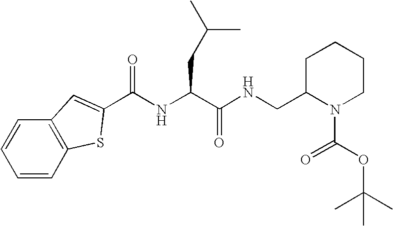

### c. 1,1-Dimethylethyl 2-({[N-(1-benzothien-2-ylcarbonyl)-L-leucyl]amino}methyl)-1-piperidinecarboxylate

EDC (0.566 g, 2.95 mmol), HOBt (0.413 g, 3.06 mmol), benzo(b)thiophene-2-carboxylic acid (0.527 g, 2.96 mmol), and triethylamine (0.57 mL, 4.07 mmol) were added to a solution of 1,1-dimethylethyl 2-[(L-leucylamino)methyl]-1-piperidinecarboxylate (0.880 g, 2.69 mmol) in CH₂Cl₂ (22 mL). The reaction was stirred at room temperature for 4 days before being diluted with CH₂Cl₂ and washed with 1N HCl, sat. NaHCO₃, and brine. The organic layer was dried over Na₂SO₄, filtered, and concentrated. Column chromatography (10-75% ethyl acetate:hexane) yielded 0.780 g (59% over 3 steps) of the title compound as a white solid: LCMS (m/z) 488.2 (M+H)⁺.

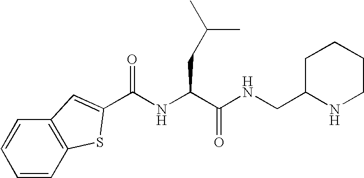

### d. N-((1S)-3-Methyl-1-{[(2-piperidinylmethyl)amino]carbonyl}butyl)-1-benzothiophene-2-carboxamide

To a solution of the product from Example 1c (0.780 g, 1.60 mmol) in methanol (16 mL) was added HCl (4.0 M in dioxane; 1.1 mL, 4.40 mmol) and the reaction stirred for 48 h. The reaction mixture was then concentrated and the crude product was carried to the next step: LCMS (m/z) 388.2 (M+H)⁺.

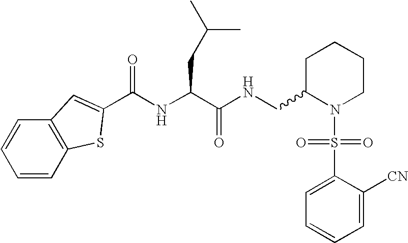

### e. N-((1S)-1-{[({1-[(2-cyanophenyl)sulfonyl]-2-piperidinyl}methyl)amino]carbonyl}-3-methylbutyl)-1-benzothiophene-2-carboxamide

Triethylamine (0.67 mL, 4.81 mmol) and 2-cyanobenzenesulfonyl chloride (0.807 g, 4.00 mmol) were added to a 0° C. solution of N-((1S)-3-methyl-1-{[(2-piperidinylmethyl)amino]carbonyl}butyl)-1-benzothiophene-2-carboxamide (0.678 g, 1.60 mmol) in CH₂Cl₂ (12 mL). The reaction was allowed to warm to room temperature and was stirred for 3 days. The reaction mixture was diluted with CH₂Cl₂ and washed with water and brine. The organic layer was dried over Na₂SO₄, filtered, and concentrated. Column chromatography (20-90% ethyl acetate:hexane) provided 0.454 g (51%) of the title compound. Separation of the mixture of diastereomers (5,5-ULMO column with 20% EtOH/Hexane as the eluent) afforded 0.298 g of the D1 isomer and 0.156 g of the D2 isomer: LCMS (m/z) 553.0 (M+H)⁺.

### Example 2

### Preparation of N-((1S)-1-{[(2-{1-[(2-Chloro-4-fluorophenyl)sulfonyl]-2-piperidinyl}ethyl)amino]carbonyl}-3-methylbutyl)-1-benzothiophene-2-carboxamide

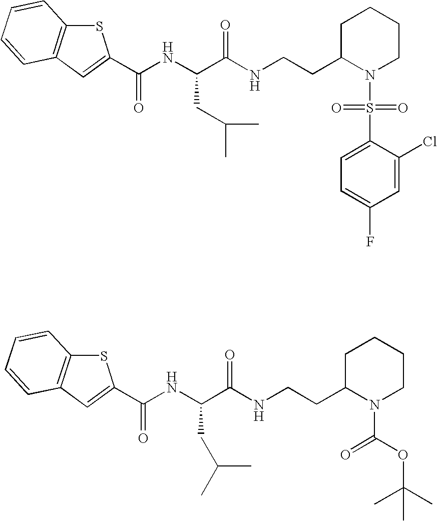

### a. 1,1-Dimethylethyl 2-(2-{[N-(1-benzothien-2-ylcarbonyl)-L-leucyl]amino}ethyl)-1-piperidinecarboxylate

To a solution of 2-(aminoethyl)-1-N-boc-piperidine (0.253 g, 1.11 mmol) in CH₂Cl₂ (8.5 mL) was added EDC (0.327 g, 1.71 mmol), HOOBt (0.035 g, 0.215 mmol), N-(1-benzothien-2-ylcarbonyl)-L-leucine (0.323 g, 1.11 mmol), and 4-methylmorpholine (0.39 mL, 3.55 mmol). The reaction mixture was stirred at room temperature for 21 hours whereupon the reaction was diluted with CH₂Cl₂ and washed with sat. NaHCO₃, 1N HCl, sat. NaHCO₃, and brine. The organic layer was dried over Na₂SO₄, filtered, and concentrated. Column chromatography (5-50% ethyl acetate:hexane) afforded 0.370 g (67%) of the title compound as a white solid: LCMS (m/z) 502.2 (M+H)⁺.

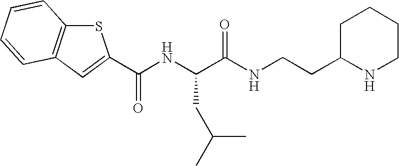

### b. N-[(1S)-3-Methyl-1-({[2-(2-piperidinyl)ethyl]amino}carbonyl)butyl]-1-benzothiophene-2-carboxamide

To a solution of the product from Example 2a (0.370 g, 0.738 mmol) in methanol (7.5 mL) was added HCl (4.0 M in dioxane; 0.52 mL, 2.08 mmol) and the reaction stirred for 4 days. The reaction mixture was then concentrated and the crude title compound was carried to the next step: LCMS (m/z) 402.2 (M+H)⁺.

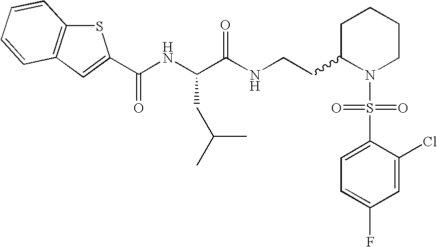

### c. N-((1S)-1-{[(2-{1-[(2-Chloro-4-fluorophenyl)sulfonyl]-2-piperidinyl}ethyl)amino]carbonyl}-3-methylbutyl)-1-benzothiophene-2-carboxamide

Triethylamine (0.40 mL, 2.87 mmol) and 2-chloro-4-fluorobenzenesulfonyl chloride (0.264 g, 1.15 mmol) were added to a solution of N-[(1S)-3-methyl-1-({[2-(2-piperidinyl)ethyl]amino}carbonyl)butyl]-1-benzothiophene-2-carboxamide (0.358 g, 0.818 mmol) in CH₂Cl₂ (8.0 mL). The reaction was stirred for 4 days whereupon it was concentrated in vacuo. Column chromatography (5-65% ethyl acetate:hexane) provided the mixture of diastereomers. Separation of the mixture (S,S-ULMO column with 2.0% EtOH/Hexane as the eluent) afforded 0.145 g of the D1 isomer and 0.156 g of the D2 isomer: LCMS (m/z) 594.2 (M+H)⁺.

### Example 3

### Preparation of N-((1S)-1-{[(2-{1-[(2,4-Dichlorophenyl)sulfonyl]-2-piperidinyl}ethyl)amino]carbonyl}-3-methylbutyl)-1-benzothiophene-2-carboxamide

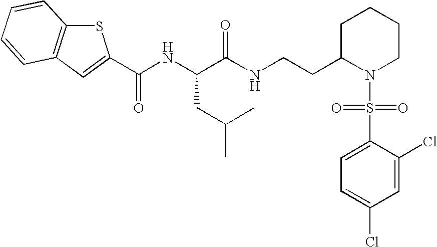

The title compound was prepared according to the general procedure described in Example 2 except substituting 2,4-dichlorobenzenesulfonyl chloride for 2-chloro-4-fluorobenzenesulfonyl chloride. Separation of the mixture of diastereomers (S,S-ULMO column with 2.0% EtOH/Hexane as the eluent) afforded 0.122 g of the D1 isomer and 0.112 g of the D2 isomer: LCMS (m/z) 610.2/612.2 (M/M+2)⁺.

### Example 4

### Preparation of N-((1S)-1-{[(2-{4-[(2,4-Dichlorophenyl)sulfonyl]-3-thiomorpholinyl}ethyl)amino]carbonyl}-3-methylbutyl)-1-benzothiophene-2-carboxamide

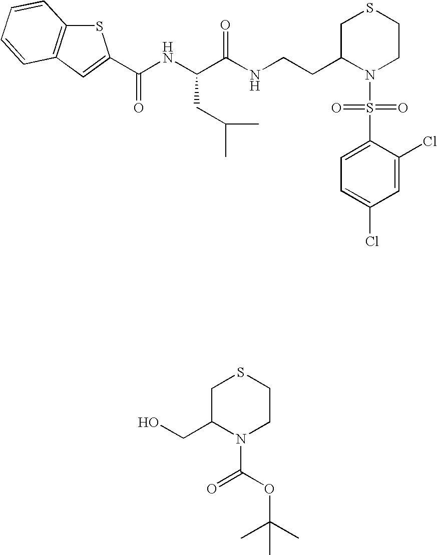

### a. 1,1-Dimethylethyl 3-(hydroxymethyl)-4-thiomorpholinecarboxylate

To a solution of 4-N-Boc-3-thiomorpholinecarboxylic acid (2.09 g, 8.44 mmol) in methanol (84 mL) was added HCl (4.0 M in dioxane; 1.07 mL, 4.28 mmol). The resulting reaction mixture was heated at reflux for 20 hours, cooled, and concentrated in vacuo. The solid residue was dissolved in THF/CH₂Cl₂ (50/30 mL) and LiBH₄ (2.0 M in THF) (10 mL, 20.0 mmol) was added dropwise over 10 minutes. The reaction mixture was stirred for 5 days before being quenched with slow addition of methanol (˜80 mL). After concentration in vacuo, the residue was dissolved in CH₂Cl₂ and washed with 1N HCl and brine. The organic layer was dried over Na₂SO₄, filtered, and concentrated to afford 1.27 g of crude product: 1H NMR (400 MHz, CDCl₃-d) δ ppm 4.31 (s, 1H) 4.06-4.18 (m, 1H) 3.69-3.75 (m, 1H) 3.60-3.67 (m, 1H) 3.40 (d, J=13.89 Hz, 1H) 3.33 (d, J=19.20 Hz, 1H) 2.72-2.84 (m, 2H) 2.27 (d, J=12.63 Hz, 1H) 1.44-1.54 (m, 10H).

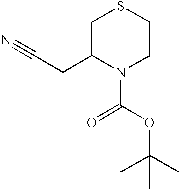

### b. 1,1-Dimethylethyl 3-(cyanomethyl)-4-thiomorpholinecarboxylate

Triethylamine (4.2 mL, 30.1 mmol) and methanesulfonyl chloride (1.4 mL, 18.0 mmol) were added to a 0° C. solution of 1,1-dimethylethyl 3-(hydroxymethyl)-4-thiomorpholinecarboxylate (0.930 g, 3.99 mmol) in CH₂Cl₂ (65 mL) under N₂. The reaction was stirred at 0° C. for 1 hour and then quenched with sat. NaHCO₃. The layers were separated and the aqueous layer was extracted two times with CH₂Cl₂. The combined organic layers were washed with brine, dried over Na₂SO₄, filtered, and concentrated. The residue was dissolved in dimethyl sulfoxide (60 mL), and potassium cyanide (1.92 g, 29.5 mmol) was added. After stirring at room temperature for 20 h, the reaction was diluted with water and brine, dried over Na₂SO₄, filtered, and concentrated to afford 0.640 g of the crude title compound which was carried to the next step.

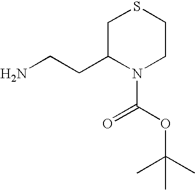

### c. 1,1-Dimethylethyl 3-(2-aminoethyl)-4-thiomorpholinecarboxylate

To a solution of 1,1-dimethylethyl 3-(cyanomethyl)-4-thiomorpholinecarboxylate (0.640 g, 2.64 mmol) in methanol (8.0 mL) was added NH₄OH (1.3 mL) and Raney Ni (˜0.880 g, washed 5× with water, 5× with ethanol, and 5× with methanol). The reaction mixture was shaken under 55 psi of H₂ for 24 h. The reaction was then filtered through Celite. The solids were washed with methanol and CH₂Cl₂, and the combined filtrate was concentrated in vacuo to afford 0.600 g (92%) of the title compound: LCMS (m/z) 247.0 (M+H)⁺.

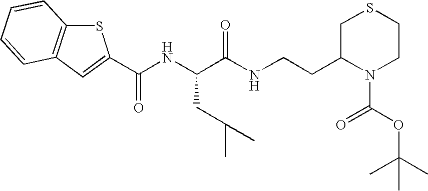

### d. 1,1-Dimethylethyl 3-(2-{[N-(1-benzothien-2-ylcarbonyl)-L-leucyl]amino}ethyl)-4-thiomorpholinecarboxylate

To a solution of 1,1-dimethylethyl 3-(2-aminoethyl)-4-thiomorpholinecarboxylate (0.600 g, 2.44 mmol) in CH₂Cl₂ (24 mL) was added EDC (0.748 g, 3.90 mmol), HOOBt (0.085 g, 0.520 mmol), N-(1-benzothien-2-ylcarbonyl)-L-leucine (0.744 g, 2.55 mmol), and 4-methylmorpholine (0.67 mL, 6.09 mmol). The reaction mixture was stirred at room temperature for 18 h whereupon the reaction was diluted with CH₂Cl₂ and washed with sat. NaHCO₃, 1N HCl, sat. NaHCO₃, and brine. The organic layer was dried over Na₂SO₄, filtered and concentrated. Column chromatography (5-66% ethyl acetate:hexane) afforded 0.670 g (53%) of the title compound as a white solid: LCMS (m/z) 520.2 (M+H)⁺.

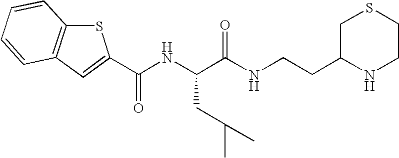

### e. N-[(1S)-3-Methyl-1-({[2-(3-thiomorpholinyl)ethyl]amino}carbonyl)butyl]-1-benzothiophene-2-carboxamide

To a solution of 1,1-dimethylethyl 3-(2-{[N-(1-benzothien-2-ylcarbonyl)-L-leucyl]amino}ethyl)-4-thiomorpholinecarboxylate (0.670 g, 1.29 mmol) in methanol (13 mL) was added HCl (4.0 M in dioxane; 1.1 mL, 4.40 mmol) and the reaction stirred for 3 days. The reaction mixture was then concentrated and the crude title compound was carried to the next step: LCMS (m/z) 420.2 (M+H)⁺.

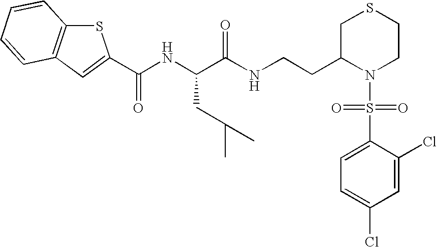

### f. N-((1S)-1-{[(2-{4-[(2,4-Dichlorophenyl)sulfonyl]-3-thiomorpholinyl}ethyl)amino]carbonyl}-3-methylbutyl)-1-benzothiophene-2-carboxamide

Triethylamine (0.54 mL, 3.87 mmol) and 2,4-dichlorobenzenesulfonyl chloride (0.496 g, 2.02 mmol) were added to a solution of N-[(1S)-3-methyl-1-({[2-(3-thiomorpholinyl)ethyl]amino}carbonyl)butyl]-1-benzothiophene-2-carboxamide (0.588 g, 1.29 mmol) in CH₂Cl₂ (13 mL). The reaction was stirred for 4 days whereupon it was concentrated in vacuo. Column chromatography (3-66% ethyl acetate:hexane) provided a mixture of diastereomers. Separation of the mixture (R,R-Whelko column with 2.0% EtOH/Hexane as the eluent) afforded 0.256 g of the D1 isomer and 0.272 g of the D2 isomer: LCMS (m/z) 628.2/630.2 (M/M+2)⁺.

### Example 5

### Preparation of N-((1S)-1-{[(2-{4-[(2,4-dichlorophenyl)sulfonyl]-1,1-dioxido-3-thiomorpholinyl}ethyl)amino]carbonyl}-3-methylbutyl)-1-benzothiophene-2-carboxamide

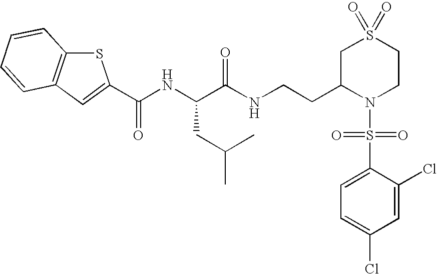

A solution of the thiomorpholine product from Example 4f (0.094 g, 0.150 mmol) in CH₂Cl₂ (1.8 mL) was cooled to 0° C. m-CPBA (77%, 0.074 g, 0.330 mmol) was added and the reaction was allowed to warm to room temperature overnight. The reaction mixture was diluted with CH₂Cl₂ and washed with 3M NaOH (3×) and brine. The organic layer was dried over Na₂SO₄, filtered and concentrated. Column chromatography (5-80% ethyl acetate/hexane) yielded 0.025 g (25%) of the title compound: LCMS (m/z) 660.0/662.2 (M/M+2)⁺.

### Example 6

The sucrose, calcium sulfate dihydrate and a TRPV4 agonist as shown in Table 2 below, are mixed and granulated in the proportions shown with a 10% gelatin solution. The wet granules are screened, dried, mixed with the starch, talc and stearic acid, screened and compressed into a tablet.

TABLE 2

| INGREDIENTS | AMOUNTS | |
|---|---|---|
| N-((1S)-1-{[({1-[(2-cyanophenyl)sulfonyl]-2- | 20 | mg |
| piperidinyl}methyl)amino]carbonyl}-3-methylbutyl)-1- | | |
| benzothiophene-2-carboxamide | | |
| calcium sulfate dihydrate | 30 | mg |
| sucrose | 4 | mg |
| starch | 2 | mg |
| talc | 1 | mg |
| stearic acid | 0.5 | mg |

1. A compound of formula I 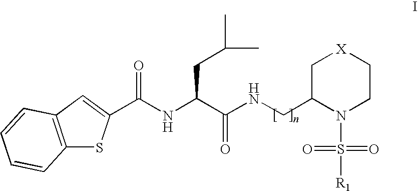
wherein
R¹ is optionally substituted aryl;
X is CH₂, S, or SO₂; and
n=1 or 2.

2. The compound of claim 1, wherein R¹ is optionally substituted phenyl.

3. The compound of claim 2, wherein the phenyl is substituted with one to three of CN, NO₂, or halogen.

4. The compound according to claim 1 selected from the group consisting of:
N-((1S)-1-{[({1-[(2-cyanophenyl)sulfonyl]-2-piperidinyl}methyl)amino]carbonyl}-3-methylbutyl)-1-benzothiophene-2-carboxamide;
N-((1S)-1-{[(2-{1-[(2-chloro-4-fluorophenyl)sulfonyl]-2-piperidinyl}ethyl)amino]carbonyl}-3-methylbutyl)-1-benzothiophene-2-carboxamide;
N-((1S)-1-{[(2-{1-[(2,4-dichlorophenyl)sulfonyl]-2-piperidinyl}ethyl)amino]carbonyl}-3-methylbutyl)-1-benzothiophene-2-carboxamide;
N-((1S)-1-{[(2-{4-[(2,4-dichlorophenyl)sulfonyl]-3-thiomorpholinyl}ethyl)amino]carbonyl}-3-methylbutyl)-1-benzothiophene-2-carboxamide; and
N-((1S)-1-{[(2-{4-[(2,4-dichlorophenyl)sulfonyl]-1,1-dioxido-3-thiomorpholinyl}ethyl)amino]carbonyl}-3-methylbutyl)-1-benzothiophene-2-carboxamide.

5. A pharmaceutical composition comprising a compound according to claim 1 or a pharmaceutically acceptable salt thereof and a pharmaceutically acceptable carrier, diluent or excipient.

6. A method of activating a TRPV4 channel receptor in a patient, comprising administering to said patient in need thereof an effective amount of a compound according to claim 1.

7. A method for treating a patient in need thereof comprising contacting at least one cell expressing a TRPV4 channel receptor of the patient with a therapeutically effective amount of a compound of formula I.

8. The method of claim 7 wherein the patient suffers from a disease affecting cartilage or matrix degradation.

9. The method of claim 8, wherein the patient is suffering from a disease or condition chosen from the group of: pain, chronic pain, neuropathic pain, postoperative pain, rheumatoid arthritis, osteoarthritis, neuralgia, neuropathies, algesia, nerve injury, ischaemia, neurodegeneration, cartilage degeneration, and inflammatory disorders.

10. The method of claim 9, wherein the patient suffers from a diseases affecting the larynx, trachea, auditory canal, intervertebral discs, ligaments, tendons, joint capsules or bone development.

11. The method of claim 10, wherein the disease is related to joint destruction.

12. The method of claim 11, wherein the patient is suffering from osteoarthritis.

13. The method of claim 11, wherein the patient is suffering from rheumatoid arthritis.

14. A pharmaceutically acceptable salt of a compound according to claim 4.
